(19) United States
(12) Patent Application Publication
(10) Pub. No.: US 2022/0009883 A1
(43) Pub. Date: Jan. 13, 2022

(54) THIOAMIDE-CONTAINING COMPOSITIONS AND METHODS OF USE THEREOF

(71) Applicant: NORIA THERAPEUTICS, INC., New York, NY (US)

(72) Inventor: Stephen DIMAGNO, Chicago, IL (US)

(21) Appl. No.: 17/293,671

(22) PCT Filed: Nov. 13, 2019
(86) PCT No.: PCT/US2019/061173

§ 371 Date: May 13, 2021

Related U.S. Application Data
(60) Provisional application No. 62/767,151, filed on Nov. 14, 2018.

(51) Int. Cl. C07C 327/44; A61K 47/54; A61K 47/62

(52) U.S. Cl. CPC C07C 327/44; A61K 47/62; A61K 47/542

(57) ABSTRACT

Provided herein are compositions and methods for preparing albumin-targeting moieties that feature a thioamide linkage. Methods to use the albumin targeting molecules to generate drugs with improved in vivo pharmacodynamics and biodistribution are described. Therapeutic compounds incorporating these thioamide linked albumin-targeting moieties are disclosed.

### RELATED APPLICATIONS

This application claims the benefit of priority to U.S. Provisional Patent Application No. 62/767,151, filed Nov. 14, 2018. The contents of this related application is hereby incorporated by reference in its entirety.

### TECHNICAL FIELD

The invention relates generally to modified drugs and more particularly to thioamide-modified drugs.

### BACKGROUND

Several strategies are currently used to extend the serum lifetime of therapeutic drugs, including alteration of peptide sequence in peptide or polypeptide drugs and secondary structure to minimize protease activity. Another approach for extending biomolecule half-life is PEGylation. Yet another approach is to introduce albumin-targeting moieties on to drugs.

### SUMMARY

This disclosure provides thioamide-modified amino acids useful as albumin-targeting moieties. These compounds offer tunable (and different) albumin binding and increased in vivo stability compared to the corresponding amide-modified compounds.

In a first aspect, disclosed herein are thioamide containing compositions as disclosed below. In embodiments they are useful, inter alia, as albumin-targeting agents.

In a first aspect, the disclosure provides a compound of Formula (I):

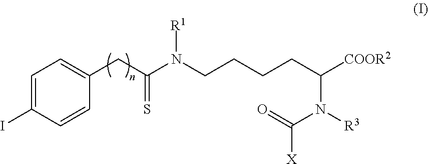

or a pharmaceutically acceptable salt thereof;
wherein:

R¹ is H, C₁-C₆ alkyl, or a protecting group;

R² is H, C₁-C₆ alkyl, or a protecting group;

R³ is H, C₁-C₆ alkyl, or a protecting group;

X is a therapeutic drug; and

n is 0, 1, 2, 3, 4, or 5.

In embodiments, n is 2 or 3.

In embodiments, the compound of Formula (I) is a compound of Formula (II):

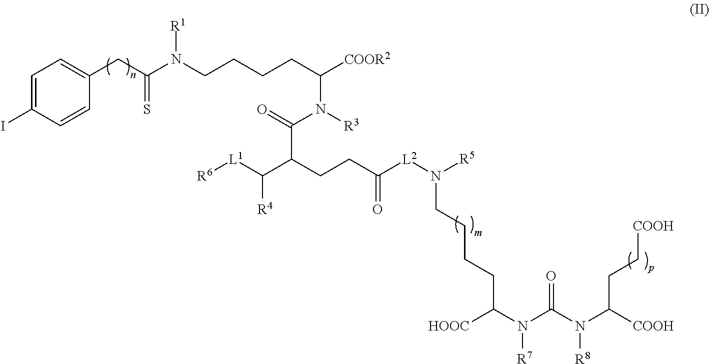

or a pharmaceutically acceptable salt thereof;
wherein:

R¹ is H, C₁-C₆ alkyl, or a protecting group;

R² is H, C₁-C₆ alkyl, or a protecting group;

R³ is H, C₁-C₆ alkyl, or a protecting group;

L¹ is a natural amino acid, an unnatural amino acid, or (X)_{q}—(Y)ᵣ—(Z)ₛ, wherein X is C₁-C₃₀ alkyl, Y is C₁₀-C₃₀ heteroaromatic, and Z is C₁-C₁₂ alkyl, wherein any of the methylene groups in the alkyl group of L¹ may be replaced with —O—, NH, or carbonyl;

R⁴ is H, C₁-C₆ alkyl, or a protecting group;

R⁵ is H, C₁-C₆ alkyl, or a protecting group;

R⁶ is a therapeutic drug or chelating agent;

R⁷ is H, C₁-C₆ alkyl, or a protecting group;

R⁸ is H, C₁-C₆ alkyl, or a protecting group;

L² is a bond, —N(R⁹)—C₁-C₁₂ alkyl-C(O)—, —N(R⁹)—C₄-C₃₀ alkylcycloalkyl-C(O)—C₇-C₃₀ alkylaryl-C(O)—, or —N(R⁹)—C₇-C₃₀ alkylaryl-C(O)NH—C₇-C₃₀ alkylaryl-C(O)NH—CH(CO₂H)—C₁-C₁₂ alkyl-NHC(O)—C₁-C₁₂ alkyl-C(O)—, wherein C₇-C₃₀ alkylaryl is optionally substituted with halo or hydroxyl;

n is 0, 1, 2, 3, 4, or 5;

m is 0, 1, 2, 3, 4, or 5;

p is 0, 1, 2, 3, 4, or 5;

q is 0 or 1;

r is 0 or 1; and

s is 0 or 1.

In embodiments, n is 3.
In embodiments, L¹ is X—Y—Z, and wherein:

X is

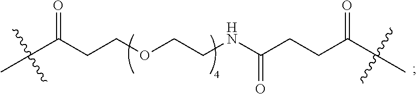

Y is

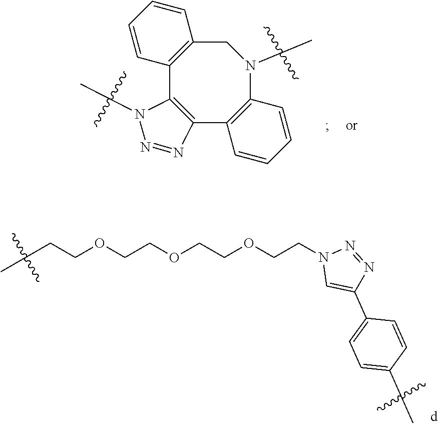 [SMILES: *N1Cc2ccccc2-c2c(nnn2*)-c2ccccc21 ; *CCOCCOCCOCCn1cc(-c2ccc(*)cc2)nn1]

Z is C₁-C₁₂ alkyl, wherein any of the methylene groups in the alkyl group may be replaced with NH or carbonyl.

In embodiments, L¹ is Z, wherein:

Z is C₁-C₁₂ alkyl, wherein any of the methylene groups in the alkyl group may be replaced with NH or carbonyl.

In embodiments, Z is

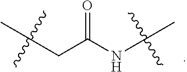 [SMILES: *CC(=O)N*]

In embodiments, the chelating agent is

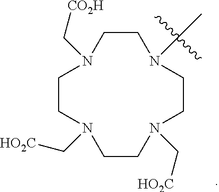 [SMILES: *N1CCN(CC(=O)O)CCN(CC(=O)O)CCN(CC(=O)O)CC1]

In embodiments, L² is —N(R⁹)—C₁-C₁₂ alkyl-C(O)—.

In embodiments, L² is

In embodiments, L² is —N(R⁹)—C₄-C₃₀ alkylcycloalkyl-C(O)—C₇-C₃₀ alkylaryl-C(O)—
In embodiments, L² is

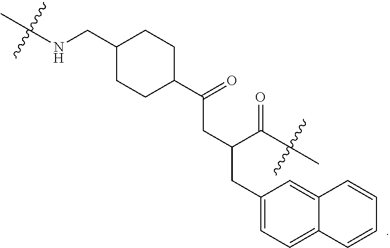 [SMILES: *NCC1CCC(C(=O)CC(Cc2ccc3ccccc3c2)C(*)=O)CC1]

In embodiments, L² is —N(R⁹)—C₇-C₃₀ alkylaryl-C(O)NH—C₇-C₃₀ alkylaryl-C(O)NH—CH(CO₂H)—C₁-C₁₂ alkyl-NHC(O)—C₁-C₁₂ alkyl-C(O)—, wherein C₇-C₃₀ alkylaryl is optionally substituted with halo or hydroxyl.

In embodiments, L² is

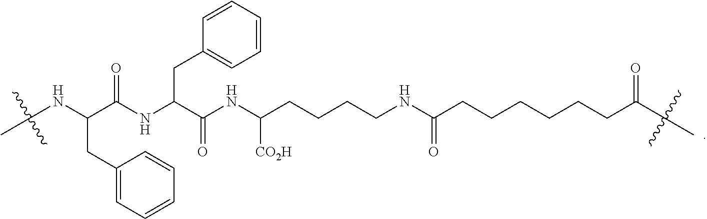 [SMILES: *NC(Cc1ccccc1)C(=O)NC(Cc1ccccc1)C(=O)NC(CCCCNC(=O)CCCCCCC(*)=O)C(=O)O]

In embodiments, L² is

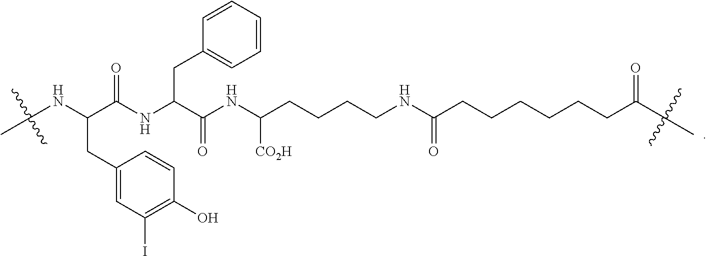 [SMILES: *NC(Cc1ccc(O)c(I)c1)C(=O)NC(Cc1ccccc1)C(=O)NC(CCCCNC(=O)CCCCCCC(*)=O)C(=O)O]

In embodiments, the compound of Formula (I) is a compound of Formula (III):

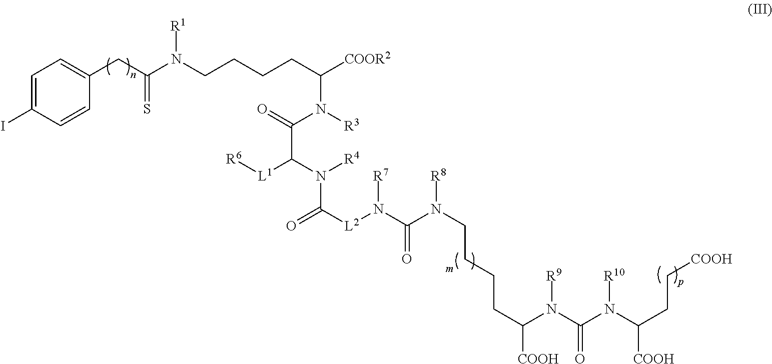

or a pharmaceutically acceptable salt thereof;
wherein:

R¹ is H, C₁-C₆ alkyl, or a protecting group;

R² is H, C₁-C₆ alkyl, or a protecting group;

R³ is H, C₁-C₆ alkyl, or a protecting group;

L¹ is a natural amino acid, an unnatural amino acid, or (X)_{q}—(Y)ᵣ—(Z)ₛ, wherein X is C₁-C₂₀ alkyl, Y is C₁₀-C₃₀ aryl, and Z is C₁-C₁₂ alkyl, wherein any of the methylene groups in the alkyl group of L¹ may be replaced with —O—, NH, carbonyl, or thiocarbonyl;

R⁴ is H, C₁-C₆ alkyl, or a protecting group;

R⁵ is H, C₁-C₆ alkyl, or a protecting group;

R⁶ is a therapeutic drug or chelating agent;

R⁷ is H, C₁-C₆ alkyl, or a protecting group;

R⁸ is H, C₁-C₆ alkyl, or a protecting group;

R⁹ is H, C₁-C₆ alkyl, or a protecting group;

R¹⁰ is H, C₁-C₆ alkyl, or a protecting group;

L² is C₁-C₃₀ alkyl-C₃-C₁₈ heteroaryl-C₆-C₁₈ aryl, wherein any of the methylene groups in the alkyl group may be replaced with —O—;

n is 0, 1, 2, 3, 4, or 5;

m is 0, 1, 2, 3, 4, or 5;

p is 0, 1, 2, 3, 4, or 5;

q is 0 or 1;

r is 0 or 1; and

S is 0 or 1.

In embodiments, n is 3.

In embodiments, L¹ is X—Y—Z, wherein:

X is

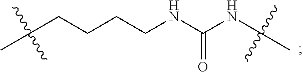 [SMILES: *CCCCNC(=O)N*]

Y is C₁₀-C₃₀ aryl; and

Z is C₁-C₁₂ alkyl, wherein any of the methylene groups in the alkyl group may be replaced with NH or carbonyl.

In embodiments, the chelating agent is

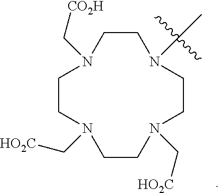 [SMILES: *N1CCN(CC(=O)O)CCN(CC(=O)O)CCN(CC(=O)O)CC1]

In embodiments, L² is

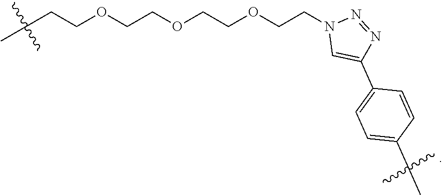 [SMILES: *CCOCCOCCOCCn1cc(-c2ccc(*)cc2)nn1]

In embodiments, compounds of Formulae (II) and (III) are provided as therapeutic drugs. In embodiments, the compounds include an albumin targeting portion, a PMSA targeting portion, and a drug or chelator portion. In embodiments, the 4-iodophenyl portion is the albumin targeting portion, the urea (or urea derivative) is the PMSA targeting portion, and R⁶ is the drug or chelator portion. The PMSA targeting portion and the drug or chelator portion may be linked to the albumin targeting group by a non-therapeutic linking moiety. In embodiments, the linking moiety may consist of a PEG chain. In other embodiments, this linking moiety is a mixture of PEG and alkyl groups. In some embodiments, this linking moiety links therapeutic drugs that do not contain PMSA-binding groups. In embodiments, therapeutic drugs are linked to the albumin binding group at the N-terminus of the lysine portion of the albumin binding group. In embodiments, this linking group is attached via a nucleophilic addition of the N-terminus of the lysine portion of the albumin targeting group to atom adjacent a leaving group on the linking moiety. In some embodiments, this leaving group is an N-hydroxy succinimide covalently attached to a carbonyl of the linking group.

In another aspect the disclosure provides a compound of Formula (IV):

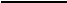

or a pharmaceutically acceptable salt thereof;
wherein:

R¹ is H, C₁-C₆ alkyl, or a protecting group;

R² is H, C₁-C₆ alkyl, or a protecting group; and

n is 0, 1, 2, 3, 4, or 5.

In an embodiment, n is 2 or 3.

### DETAILED DESCRIPTION

Disclosed herein are thioamide-containing compositions useful, inter alia, as albumin binding agents for imaging and therapeutic modalities

In embodiments, the thioamide-containing compositions bind with high affinity to prostate-specific membrane antigens (PSMA), analogous to the PSMA binding compounds disclosed in WO2018/098390 and WO2013/028664, whose contents are incorporated herein by reference in their entirety.

In embodiments, compositions according to the disclosure can be used in methods analogous to those taught for the thioamide-containing compounds disclosed in US2018/0066298, whose contents are incorporated herein by reference in their entirety.

In embodiments, the disclosure provides a modified drug comprising lysine or ornithine and an albumin targeting group, wherein the lysine or ornithine is linked to the albumin-targeting group by a thioamide moiety (i.e., a thioamide linkage). In certain embodiments, the thioamide linkages are more stable to in vivo hydrolysis, as compared to amide linkages. In certain embodiments, the thioamide linkages are more stable to peptidase activity, as compared to amide linkages. In certain embodiments, the compound has greater in vivo stability than a corresponding compound wherein a lysine or ornithine is linked to an albumin-targeting group by an amide moiety. The artisan can link the drug to the lysine or ornithine moiety using techniques known in the art.

### Definitions

A “therapeutically effective amount” of a compound or a pharmaceutical composition refers to an amount effective to prevent, inhibit, lessen, or treat the symptoms of a particular disorder or disease.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. The compositions can be incorporated into particulate preparations of polymeric compounds such as polylactic acid, polyglycolic acid, etc., or into liposomes or micelles. Such compositions may influence the physical state, stability, rate of in vivo release, and rate of in vivo clearance of components of a pharmaceutical composition of the present invention. The pharmaceutical composition of the present invention can be prepared, for example, in liquid form, or can be in dried powder form (e.g., lyophilized). Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, 20th Edition, (Lippincott, Williams and Wilkins), 2000; Liberman, et al., Eds., Pharmaceutical Dosage Forms, Marcel Decker, New York, N.Y., 1980; and Kibbe, et al., Eds., Handbook of Pharmaceutical Excipients (3.sup.rd Ed.), American Pharmaceutical Association, Washington, 1999.

“Enhanced binding” means the binding between at least two molecules, wherein at least one molecule is changed from its native state so that the binding affinity is greater between the two molecules. For example, molecule A may not bind or weakly bind to molecule B, but when molecule A is modified (A′), such as by the introduction of a non-natural amino acid having an affinity tag added thereto, molecule A′ binds with greater affinity for molecule B. In the methods and compositions of the invention, molecule A′ is a polypeptide modified with a non-natural amino acid with an albumin-binding tag, such as N_{ε}-(4-(4-iodophenyl)butanoyl)lysine, and molecule B is albumin, such as human serum albumin. Enhanced binding can be measured using a variety of techniques, including affinity determination by surface plasmon resonance and direct binding assays

The terms “isolated,” “purified,” or “biologically pure” refer to material that is substantially or essentially free from components that normally accompany it as found in its native state.

The term “subject” refers to a mammal. A subject therefore refers to, for example, dogs, cats, horses, cows, pigs, guinea pigs, and the like. Preferably the subject is a human. When the subject is a human, the subject may be referred to herein as a patient.

Various methodologies of the instant invention include steps that involve comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing a methodology, as described herein.

A “non-natural” amino acid as used herein means an amino acid either not occurring in nature (novel and synthesized amino acids), or occurring in nature but not naturally occurring within proteins (natural but non-proteinogenic amino acids).

The term “thioyl” refers to a divalent chemical functional group that is conventionally represented as a carbon atom having a double bond to a sulfur atom.

A “drug” as used herein, means a pharmaceutical formulation containing at least one pharmaceutically active compound. In one embodiment the pharmaceutically active compound has a molecular weight up to 1500 Da and/or is a peptide, a protein, a polysaccharide, a vaccine, a DNA, an RNA, an enzyme, an antibody or a fragment thereof, a hormone or an oligonucleotide, or a mixture of the above-mentioned pharmaceutically active compound.

### Compounds Containing Thioamide Linkages

In an aspect, compounds are disclosed herein that comprise albumin targeting groups. An “albumin targeting group,” “albumin targeting molecule,” or “albumin targeting tag” is a small molecule that is incorporated into a second molecule, such as a polypeptide, such that the small molecule directs the second molecule to associate with albumin, in vitro or preferably in vivo. Such association comprises a binding interaction between the albumin and the albumin targeting tag.

The term “drug” as used herein, means a pharmaceutical formulation containing at least one pharmaceutically active compound. In one embodiment the pharmaceutically active compound has a molecular weight up to 1500 Da and/or is a peptide, a protein, a polysaccharide, a vaccine, a DNA, an RNA, an enzyme, an antibody or a fragment thereof, a hormone or an oligonucleotide, or a mixture of the above-mentioned pharmaceutically active compound.

Drugs can include, e.g., anti-inflammatory drugs disclosed in embodiments, the drug is an anti-inflammatory agent, e.g., an anti-inflammatory agent disclosed in U.S. Ser. No. 12/351,417. As used herein, an “anti-inflammatory therapeutic agent” refers to compounds for the treatment of an inflammatory disease or the symptoms associated therewith. Anti-inflammatory therapeutic agents include, without limitation, non-steroidal anti-inflammatory drugs (NSAIDs; e.g., aspirin, ibuprofen, naproxen, methyl salicylate, diflunisal, indomethacin, sulindac, diclofenac, ketoprofen, ketorolac, carprofen, fenoprofen, mefenamic acid, piroxicam, meloxicam, methotrexate, celecoxib, valdecoxib, parecoxib, etoricoxib, and nimesulide), corticosteroids (e.g., prednisone, betamethasone, budesonide, cortisone, dexamethasone, hydrocortisone, methylprednisolone, prednisolone, tramcinolone, and fluticasone), rapamycin, rho-kinase inhibitors, viral CC-chemokine inhibitor (vCCIs), glucocorticoids, steroids, beta-agonists, anticholinergic agents, methyl xanthines, sulphasalazine, dapsone, psoralens, proteins, peptides, DMARDs, glucocorticoids, methotrexate, sulfasalazine, chloriquine, gold, gold salt, copper, copper salt, penicillamine, D-penicillamine, cyclosporine, lipoxins, resolving, and protecting.

In a particular embodiment, the anti-inflammatory therapeutic agent is selected from the group consisting of proteins, peptides, NSAIDs, DMARDs, glucocorticoids, methotrexate, sulfasalazine, chloriquine, gold, gold salt, copper, copper salt, penicillamine, D-penicillamine, cyclosporine, and dexamethasone. Anti-inflammatory therapeutic agents are also provided in The Pharmacological Basis of Therapeutics, 10^{th} ed., Gilman et al., eds., McGraw-Hill Press (2001) and Remington's Pharmaceutical Science's, 18th ed. Easton: Mack Publishing Co. (1990).

Other drugs include, e.g., an analgesic agent, an antialopecia agent, an antianginal agent, an antibacterial agent, an antidepressant agent, an antifungal agent, an antihypertensive agent, an antineoplastic agent, an antipyretic agent, an antipsychotic agent, an anxiolytic agent, a bronchodilator agent, a glucocorticoid, an immunosuppressant agent, acetylsalicylic acid, alpha-atrial natriuretic peptide, arginine vasopressin, atropine, augmerosen, atorvastatin, avastin, calcitonins, chlorhexidine, chorionic gonadotropins, corticotropin, desmopressin, epibatidine, erbitux, exenatide, herceptin, humira, humulin, ketoconazole, lanreotide, lutropin alpha, metoprolol, minoxidil, nesiritide, octreotide, paclitaxel, paracetamol, pegaptanib, recombinant follicle stimulating hormone, a recombinant growth factor, remicade, rituxan, sermorelin, somatotropin, a taxane derivative, taxol, teriparatide acetate, thyrotropin, triclosan, urofollitropin, xolair, actinomycin D, albendazole, aldosterone, alprazolam, amiodarone, amitriptyline, amprenavir, asimadoline, atorvastatin, bunitrolol, buspirone, camptothecin, carbamazepine, carvedilol, celiprolol, cyclosporine A, cimetidine, clotrimazole, colchicine, cortisone, daunorubicin, debrisoquine, diazepam, digitoxin, digoxin, diltiazem, docetaxel, domperidone, doxorubicin, efavirenz, epirubicin, erythromycin, ergotamine, estradiol, estradiol glucuronide, erlotinib, etoposide, phenytoin, fentanyl, felodipine, phenothiazines, fexofenadine, fluoroquinolones, fluorouracil, FK-506, gentamicin, griseofulvin, imatinib, indinavir, itraconazole, ivermectin, ketoconazole, kaempferol, levofloxacin, lidocaine, loperamide, losartan, lovastatin, mebendazole, methylprednisolone, methotrexate, mibefradil, midazolam, nisoldipine, morphine, nelfinavir, nicardipine, nitrendipine, nifedipine, ondansetron, paclitaxel, pentazocine, praziquantel, prednisolone, prednisone, quercetin, quinidine, ranitidine, rapamycin, rifabutin, rifampicin, ritonavir, saquinavir, sirolimus, sulfamethizole, tacrolimus, tamoxifen, talinolol, teniposide, terfenadine, tetracycline, topotecan, triamcinolone, valspodar, verapamil, vinblastine, vincristine, vindesine, zopiclone, a herbicide, an insecticide, a fungicide, an anti-aging product, an anti-acne product, a facial care product, a pigmented cosmetic, a cosmetical, a personal care product, a product for sunscreen/suncare, a product for tooth-cleaners, toothpastes, or rinses, a product for shampooes, a perfume, a hair products, a food additive, an essential oil, Mentha piperita oil, Thyme oil, cinnamon oil, eugenol, lemon oil, curcumin, folic acid, 4-aminobenzoic acid, niacin or vitamin B3, pantothenic acid or vitamin B5, thiamine monophosphate, thiamine pyrophosphate, thiamine triphosphate, ascorbic acid, pteroylpolyglutamic acids, folinic acid, nicotinic acid, hyaluronic acid, thioctic acid, p-coumaric acid, caffeic acid, a vitamin of the A, D, E, K families and derivatives thereof, a phospholipid, a carotenoid, a fatty acid, an omega-3 fatty acid, cod liver oil, linolenic acid, an amino acid, a phytostanol, a phytosterol, a polyphenol, chlorhexidine, bovine serum albumin, and mixtures thereof.

In embodiments, the chelator is 1,4,7,10-tetraazacyclododecane-1,4,7,10-tetraacetic acid (DOTA), 1,4,7-triaza-cyclo-nonane-1,4,7-triacetic acid (NOTA), 1,4,7-triazacyclononane-1,4-diacetic acid (NODA), or diethylenetriaminepentaacetic acid (DTPA);

The selection of a drug for targeting to albumin can be determined by one of skill in the art. For polypeptide therapeutics, its function would benefit from binding to serum albumin to, for example, increase the serum half-life of the therapeutic polypeptide. in those embodiments directed to albumin targeting of polypeptides. General examples of therapeutic polypeptides include, but are not limited to, antibodies, chimeric antibodies, monoclonal antibodies, single chain antibodies, Fab, Fab′, F(ab′)2, Fv, and scF, Fc fusions, anticoagulants, blood factors, bone morphogenetic proteins, engineered protein scaffolds, enzymes, growth factors, hormones, interferons, interleukins, and thrombolytics. Other examples of therapeutic peptides include: salmon calcitonin; β-interferon; □ interferon; veraglucerase-; taliglucerase-□ □; glucarpidase (e.g., for treatment of methotrexate toxicity); elosulfase-□ (e.g., for treatment of Morquio syndrome); aldesleukin; anakinra; insulin lispro; uricase (e.g., for treatment of gouty tophi); palifermin.

Drug-containing thioamide compositions may be administered by any desirable and appropriate means. For in vivo delivery (i.e., to a subject having arthritis or other inflammatory diseases), it is preferred that the delivery system be biocompatible and preferably biodegradable and non-immunogenic. In addition, it is desirable to deliver a therapeutically effective amount of a compound in a physiologically acceptable carrier. Injection into an individual may occur subcutaneous, intravenously, intramuscularly, intraperitoneal, intraarticular or, for example, directly into a localized area. Alternatively, in vivo delivery may be accomplished by use of a syrup, an elixir, a liquid, a tablet, a pill, a time-release capsule, an aerosol, a transdermal patch, an injection, a drip, an ointment, etc.

### EXAMPLES

The following examples are offered for illustrative purposes, and are not intended to limit the disclosure in any manner. Those of skill in the art will readily recognize a variety of noncritical parameters which can be changed or modified to yield essentially the same results.

### Example 1. Synthesis of Thioamide Containing Compositions

### A Thioamide-Containing Composition is Prepared Using the Following Synthetic Scheme:

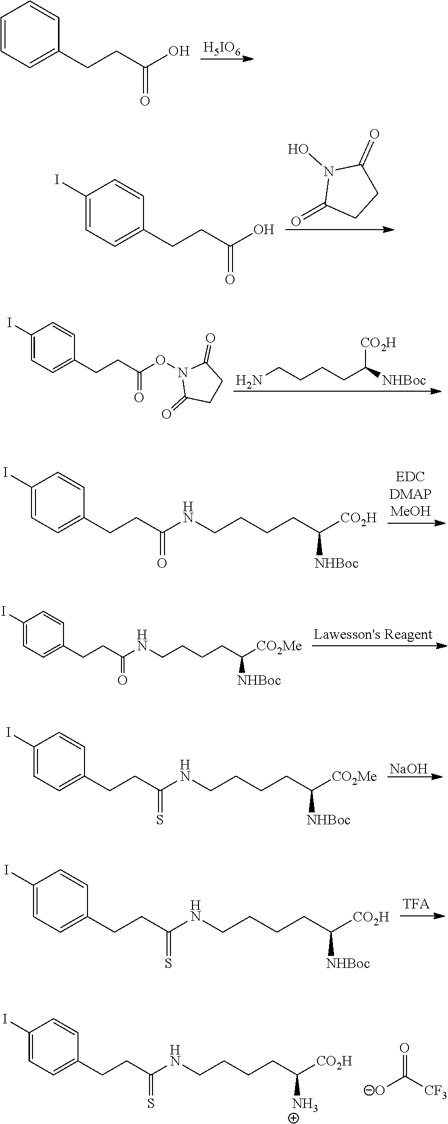

### Synthesis of 3-(4-Iodophenyl)propanoic Acid

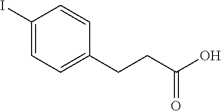 [SMILES: O=C(O)CCc1ccc(I)cc1]

To a mixture of 3-phenylpropanoic acid (20.0 g, 133.18 mmol), H5106 (6.18 g, 26.68 mmol), iodine (14.54 g, 57.3 mmol), 10 M H₂SO₄ (5.0 mL), water (36 mL) and acetic acid (166 mL) was added, and the mixture was heated at 70° C. for 19 h. The reaction mixture was cooled and evaporated to dryness. The residue was dissolved in EtOAc (300 mL) and washed with Na₂S₂O₃ (2×200 mL), brine (2×200 mL), dried over Na₂SO₄, filtered, and evaporated to leave a yellow solid. The crude product was precipitated from EtOAc/hexane at 0° C. to afford the title compound as a light-yellow solid (15.0 g, 42%). ¹H NMR (400 MHz, CDCl₃) δ 10.7 (s(br), 1H), 7.63 (d, J=8.2 Hz, 2H), 6.99 (d, J=8.2 Hz, 2H), 2.92 (t, J=7.6 Hz, 2H), 2.68 (t, J=7.6 Hz, 2H).

### Synthesis of 2,5-Dioxopyrrolidin-1-yl-3-(4-iodophenyl)propanoate

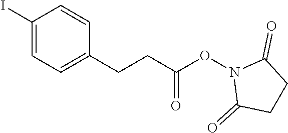 [SMILES: O=C(CCc1ccc(I)cc1)ON1C(=O)CCC1=O]

Under a nitrogen atmosphere, 3-(4-iodophenyl)propanoic acid (9.40 g, 34 mmol) was dissolved in dichloromethane (100 mL), and N-hydroxysuccinimide (6.0 g, 51.1 mmol, 1.5 eq) was added. The mixture was cooled 0° C. before a dichloromethane solution of dicyclohexylcarbodiimide (DCC, 10.55 g, 51.1 mmol, 1.5 eq) was added dropwise. The reaction mixture was stirred for 6 h at room temperature, filtered, and the filtrate was evaporated to dryness. The residue was purified by silica gel column chromatography by straight (CH₂Cl₂/methanol). Evaporation of the appropriate fractions gave the titled compound (10.98 g, 86.5%) as a white solid. ¹H NMR (400 MHz, DMSO-d6) δ 7.65 (d, J=8.2 Hz, 2H), 7.14 (d, J=8.2 Hz, 2H), 3.01 (t, J=6.84 Hz, 2H), 2.91 (d, J=6.96 Hz, 2H), 2.81 (s, 4H).

### Synthesis of N²-(tert-butoxycarbonyl)-N⁶-(4-(4′-iodophenyl)propanoyl)-L-lysine

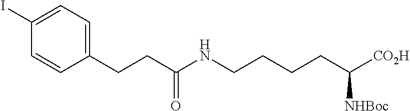 [SMILES: CC(C)(C)OC(=O)N[C@@H](CCCCNC(=O)CCc1ccc(I)cc1)C(=O)O]

Under an atmosphere of purified nitrogen, 2,5-dioxopyrrolidin-1-yl-3-(4-iodophenyl)propanoate (2 g, 5.36 mmol, 1.06 eq) dissolved in 20 mL was treated with N²-(tert-butoxycarbonyl)-L-lysine (1.35 g, 5.04 mmol). The mixture was cooled to 0° C. and DIPEA (0.88 ml, 5.04 mmol) was added dropwise. After 5 h at room temperature the solvents were removed, and the title compound was isolated by extraction from basic, followed by pH 3.5 water and CH₂Cl₂. The final organic extracts were combined, dried over Na₂SO₄, filtered, and evaporated to leave a colorless solid (80.0%). ¹H NMR (400 MHz, CD₃CN) δ 7.65 (d, J=8.3 Hz, 2H), 7.04 (d, J=8.3 Hz, 2H), 6.36 (t, 1H, NH), 5.62 (d, J=7.1 Hz, 1H, NH), 4.02 (dd, 1H), 3.11 (m, 2H), 2.85 (t, J=7.44 Hz, 2H), 2.39 (t, J=7.80 Hz, 2H), 1.43 (s, 9H).

### Synthesis of Methyl N²-(tert-butoxycarbonyl)-N⁶-(3-(4-iodophenyl)propanoyl)-L-lysinate

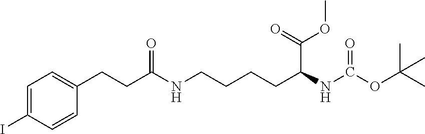 [SMILES: COC(=O)[C@H](CCCCNC(=O)CCc1ccc(I)cc1)NC(=O)OC(C)(C)C]

To a solution of N²-(tert-butoxycarbonyl)-N⁶-(3-(4-iodophenyl)propanoyl)-L-lysine (0.76 g, 1.506 mmol) in dichloromethane (10 mL), 1-ethyl-3-(3-dimethylaminopropyl)carbodiimide (EDC) (0.28 g, 1.506 mmol) added. The mixture was cooled to 0° C. and 4-dimethylaminopyridine (DMAP) (0.02 g, 0.1506 mmol, 0.1 eq) followed by dry MeOH (0.13 mL, 3.012 mmol, 2 eq) were added. The reaction mixture was allowed to stir at room temperature overnight before the solvent was evaporated and the residue was purified by silica gel column chromatography (eluant=10% methanol in dichloromethane). Evaporation of the appropriate fractions gave the title compound, isolated as a colorless solid (0.52 g, 66.6%). ¹H NMR (400 MHz, CD₃CN) δ 7.65 (d, J=8.28 Hz, 2H), 7.04 (d, J=8.28 Hz, 2H), 6.34 (t, 1H, NH), 5.65 (d, J=7.04 Hz, 1H, NH), 4.06 (dd, 1H), 3.68 (s, 3H), 3.10 (m, 2H), 2.85 (t, J=7.48 Hz, 2H), 2.85 (t, J=7.72 Hz, 2H), 1.72 (m, 1H), 1.62 (m, 1H), 1.42 (s, 9H) 1.30 (m, 2H).

### Synthesis of Methyl N²-(tert-butoxycarbonyl)-N⁶-(3-(4-iodophenyl)propanethioyl)-L-lysinate

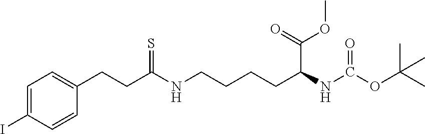 [SMILES: COC(=O)[C@H](CCCCNC(=S)CCc1ccc(I)cc1)NC(=O)OC(C)(C)C]

To 20 mL of dry toluene under argon, methyl N²-(tert-butoxycarbonyl)-N⁶-(3-(4-iodophenyl)propanethioyl)-L-lysinate (0.49 g, 0.94 mmol) and Lawesson's reagent (0.095 g, 0.24 mmol) were dissolved and allowed to stir overnight at 70° C. The solvent was evaporated, and the residue was purified by silica gel column chromatography (MeOH gradient 0-10% in CH₂Cl₂). The selected fractions were evaporated, and the product was dried under dynamic vacuum. Yield: 40%. ¹H NMR (400 MHz, CD₃CN) δ 8.29 (S, 1H), 7.65 (d, J=8.12 Hz, 2H), 7.05 (d, J=8.16 Hz, 2H), 5.57 (d, 1H, NH), 4.09 (m, 1H), 3.69 (s, 3H), 3.51 (q, J=6.08, 2H), 3.01 (t, J=7.60, Hz, 2H), 2.84 (t, J=7.64, Hz, 2H), 1.74 (m, 1H), 1.62 (m, 1H), 1.52 (m, 2H), 1.42 (s, 9H), 1.31 (m, 2H).

### Synthesis of N²-(tert-Butoxycarbonyl)-N⁶-(3-(4-iodophenyl)propanethioyl)-L-lysine

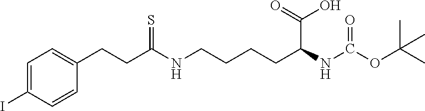 [SMILES: CC(C)(C)OC(=O)N[C@@H](CCCCNC(=S)CCc1ccc(I)cc1)C(=O)O]

Methyl N²-(tert-butoxycarbonyl)-N⁶-(3-(4-iodophenyl)propanethioyl)-L-lysinate (0.40 g, 0.74 mmol) was dissolved in i-PrOH (13 mL) and added to a mixture of NaOH (0.051 g, 0.89 mmol) and CaCl₂ (1.47 g, 13.3 mmol) in H₂O (5 mL). The mixture was stirred 4 h, at room temperature, neutralized with 1 M AcOH, and the excess of i-PrOH was evaporated. Saturated aqueous NaCl was added to the residue and the mixture was extracted three times with dichloromethane. The organic phases were combined, dried over Na₂SO₄, filtered, and evaporated. The residue was purified by silica gel column chromatography in (CHCl₂, methanol 0-10% gradient). Evaporation of the selected fractions gave the title compound (0.38 g, 100%) isolated as a yellow solid. ¹H NMR (400 MHz, MeOD) δ 7.61 (d, J=8.28 Hz, 2H), 7.03 (d, J=8.28 Hz, 2H), 4.00 (m, 1H), 3.53 (t, J=7.16 Hz, 2H), 3.01 (t, J=7.2 Hz, 2H), 2.85 (t, J=8.00 Hz, 2H), 1.82 (m, 1H), 1.66 (m, 1H), 1.57 (m, 2H), 1.46 (s, 9H), 1.37 (q, J=7.68 Hz, 2H).

### Synthesis of N⁶-(3-(4-iodophenyl)propanethioyl)-L-lysine, Trifluoroacetate Salt

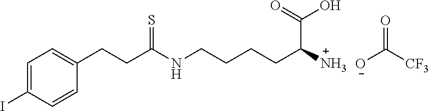 [SMILES: [NH3+][C@@H](CCCCNC(=S)CCc1ccc(I)cc1)C(=O)O ; O=C([O-])C(F)(F)F]

N²-(tert-Butoxycarbonyl)-N⁶-(3-(4-iodophenyl)propanethioyl)-L-lysine was dissolved in a solution of trifluoroacetic acid (2 mL) in CH₂Cl₂. The solution was allowed to stir at room temperature for 5 h and the solvent was evaporated. This material was used with further purification. ¹H NMR (400 MHz, MeOD) δ 7.61 (d, J=8.28 Hz, 2H), 7.03 (d, J=8.28 Hz, 2H), 3.96 (t, J=6.4 Hz, 1H), 3.57 (td, J=7.2 Hz, J=2.2 Hz, 2H), 3.04 (t, J=8.60 Hz, 2H), 2.86 (t, J=7.92 Hz, 2H), 1.95 (m, 2H), 1.61 (m, 2H), 1.40 (m, 2H).

### Example 2: Alternative Synthesis of Thioamide Containing Compositions

An alternative scheme to synthesize a thioamide containing composition is shown below:

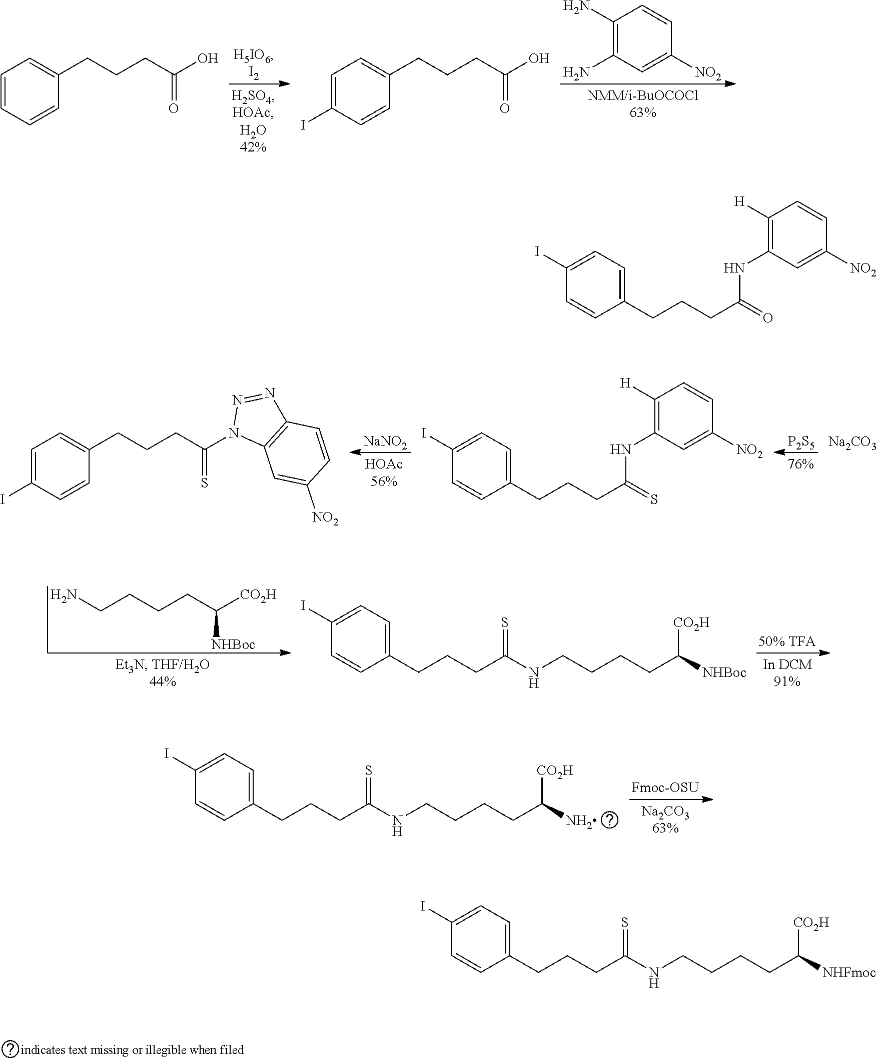

### Synthesis of 4-(4-Iodophenyl)butanoic Acid

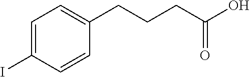 [SMILES: O=C(O)CCCc1ccc(I)cc1]

A mixture of 4-phenylbutanoic acid (20.0 g, 121.8 mmol), H₅IO₆ (5.56 g, 24.4 mmol), iodine (13.30 g, 52.4 mmol), 10 M H₂SO₄ (5.0 mL), water (36 mL) and acetic acid (166 mL) was heated at 70° C. for 19 h. The reaction mixture was cooled, and the solvents were concentrated under reduced pressure. The residue was dissolved in EtOAc (300 mL) and washed with Na₂S₂O₃ (2×200 mL), and brine (2×200 mL). The organic phase was separated, dried over Na₂SO₄, filtered, and evaporated to leave a yellow solid. The crude product was precipitated from EtOAc/hexane at 0° C. to afford product as light-yellow solid (15.0 g, 42%). ¹H NMR (400 MHz, CDCl₃) δ 11.0 (s (broad), 1H), 7.61 (d, J=8.4 Hz, 2H), 6.95 (d, J=8.0 Hz, 2H), 2.63 (t, J=7.6 Hz, 2H), 2.38 (t, J=7.6 Hz, 2H), 1.95 (A₂B₂, t, 2H).

### Synthesis of N-(2-amino-5-nitrophenyl)-4-(4-iodophenyl)butanamide

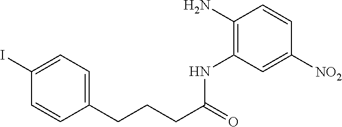 [SMILES: Nc1ccc([N+](=O)[O-])cc1NC(=O)CCCc1ccc(I)cc1]

Under a nitrogen atmosphere a solution of 4-(4-iodophenyl)butanoic acid (11.60 g, 40 mmol) in THF (200 mL) was cooled to −20° C. and treated sequentially with N-methylmorpholine (NMM) (8.8 mL, 80 mmol, 2.0 eq) and isobutyl chloroformate (5.2 mL, 40 mmol, 1.0 eq). The reaction mixture was allowed to stir for 30 min, a solution of 4-nitro-1,2-phenylenediamine (6.12 g, 40 mmol, 1.0 equiv) in THF (100 mL) was added, and the mixture was kept at −20° C. for a further 1.5 h and at 23° C. for the following 15 h. The mixture was filtered, and the filtrate was evaporated to dryness under reduced pressure. The residue was dissolved in EtOAc (300 mL) and washed with aqueous solutions of 1 M NaH₂PO₄ (2×100 mL), saturated brine (2×100 mL), saturated NaHCO₃ (2×100 mL), and saturated NaCl (2×100 mL). The organic layer was separated, dried over Na₂SO₄, and evaporated to dryness. The crude product was sonicated in EtOAc to form a solid. The EtOAc was decanted away and the remaining solid was filtered and dried in vacuo to yield the title compound (10.77 g, 63%) as a yellow-brown solid. ¹H NMR (400 MHz, DMSO-d₆) δ 9.13 (s, 1H), 8.25 (d, J=2.4 Hz, 1H), 7.83 (dd, J=8.8, 2.4 Hz, 1H), 7.64 (d, J=8.0 Hz, 2H), 7.06 (d, J=8.0 Hz, 2H), 6.75 (d, J=8.8 Hz, 1H), 6.44 (s (broad), 2H), 2.60 (t, J=7.2 Hz, 2H), 2.35 (t, J=7.2 Hz, 2H), 1.89 (A₂B₂, t, 2H).

### Synthesis of N-(2-Amino-5-nitrophenyl)-4-(4-iodophenyl)butanethioamide

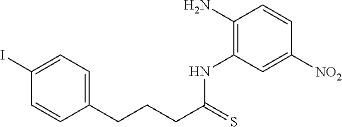 [SMILES: Nc1ccc([N+](=O)[O-])cc1NC(=S)CCCc1ccc(I)cc1]

Under a flow of nitrogen, P₂S₅ (4.44 g, 20 mmol, 1.0 eq) was added to a suspension of Na₂CO₃ (1.08 g, 10 mmol, 0.5 eq) in THF (200 mL) at 23° C. After 1 h, the mixture was cooled to 0° C. before a solution of N-(2-amino-5-nitrophenyl)-4-(4-iodophenyl)butanamide (8.50 g, 20 mmol) in THF (100 mL) was introduced. The stirred mixture was held at 0° C. for 2 h and at 23° C. for an additional hour. The solvent was evaporated under reduced pressure. The residue was dissolved in EtOAc (200 mL), washed with 5% aqueous NaHCO₃ (2×100 mL) and the aqueous phase was extracted with EtOAc (100 mL) once. The combined organic phases were dried over Na₂SO₄, filtered and evaporated to dryness. The residue was sonicated in EtOAc and the remaining solid was filtered and dried in vacuo to yield the title compound (6.89 g, 76%) as a yellow solid. ¹H NMR (400 MHz, DMSO-d₆) δ 11.0 (s (broad), 1H), 7.95 (d, J=2.4 Hz, 1H), 7.92 (dd, J=8.8, 2.4 Hz, 1H), 7.65 (d, J=8.0 Hz, 2H), 7.07 (d, J=8.0 Hz, 2H), 6.78 (d, J=8.8 Hz, 1H), 6.50 (s (broad), 2H), 2.76 (t, J=7.6 Hz, 2H), 2.64 (t, J=7.6 Hz, 2H), 2.08 (A₂B₂, t, 2H).

### Synthesis of 4-(4-Iodophenyl)-1-(6-nitro-1H-benzo[d][1,2,3]triazol-1-yl)butane-1-thione

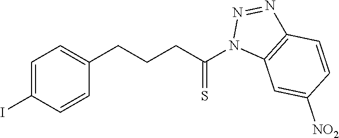 [SMILES: O=[N+]([O-])c1ccc2nnn(C(=S)CCCc3ccc(I)cc3)c2c1]

A solution of N-(2-amino-5-nitrophenyl)-4-(4-iodophenyl)butanethioamide (5.68 g, 12.8 mmol) in 95% glacial acetic acid (300 mL) was cooled to 0° C. NaNO₂ (1.32 g, 19.2 mmol, 1.5 eq) was added in portions over 20 min to the stirred mixture. After 30 min, the precipitated product was filtered, washed with water and the filtrate was extracted with EtOAc (2×150 mL). The combined organic phases were washed successively with H₂O (3×100 mL), saturated NaHCO₃ (2×100 mL), and brine (2×100 mL). The organic layer was separated, dried of Na₂SO₄, filtered, and evaporated to dryness under reduced pressure. The remaining solid was sonicated in a small amount of EtOAc (5 mL). The EtOAc was decanted away and the remaining solid was filtered. The remaining product (yellow solid, 3.23 g, 56%) was dried in vacuo. ¹H NMR (400 MHz, CDCl₃) δ 9.71 (d, J=1.6 Hz, 1H), 8.45 (dd, J=8.8, 2.0 Hz, 1H), 8.31 (d, J=8.8 Hz, 1H), 7.59 (d, J=8.0 Hz, 2H), 6.98 (d, J=8.0 Hz, 2H), 3.80 (t, J=7.6 Hz, 2H), 2.79 (t, J=7.6 Hz, 2H), 2.33 (A₂B₂, t, 2H).

### Synthesis of N²-(tert-butoxycarbonyl)-N⁶-(4-(4-iodophenyl)butanethioyl)-L-lysine

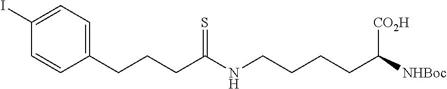 [SMILES: CC(C)(C)OC(=O)N[C@@H](CCCCNC(=S)CCCc1ccc(I)cc1)C(=O)O]

To a cooled solution (0° C.) of thioacylating reagent (5 mmol, 2.26 g) in 75 mL of THF was added dropwise a solution of Boc-Lys-OH (5 mmol, 1.23 g) and triethylamine in 15 mL of THF and 3.0 mL of H₂O over a period of 1 h. After the addition was complete, the mixture was then allowed to stir overnight at room temperature. The mixture was extracted with EtOAc, dried over sodium sulfate, filtered and concentrated under reduced pressure. The residue was purified by silica gel column chromatography (hexane/EtOAc, 1/1, followed by 100% MeOH) to afford the title compound (1.18 g) in 44% yield.

### Synthesis of N⁶-(4-(4-iodophenyl)butanethioyl)-L-lysine Trifluoroacetic Acid Salt

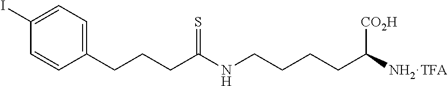

To a solution of N²-(tert-butoxycarbonyl)-N⁶-(4-(4-iodophenyl)butanethioyl)-L-lysine (1.05 g, 2 mmol) in dry CH₂Cl₂ (2 mL) was added TFA (2.0 mL), and the reaction mixture was allowed to stir for 4 h at room temperature. The solvents were evaporated under reduced pressure and the remaining solid and dried under dynamic vacuum overnight. The residue was dissolved ethyl acetate (10.0 mL) and allowed to precipitate over the course of 12 h. The precipitate was filtered and dried in vacuum to give the title amino acid (0.97 g, 91%) as gray solid. ¹H NMR (400 MHz, MeOH-d₄) δ 7.61 (d, J=8.0 Hz, 2H), 7.00 (d, J=8.0 Hz, 2H), 3.97 (t, J=6.4 Hz, 1H), 3.63 (t, J=7.6 Hz, 2H), 2.67-2.52 (m, 3H), 2.11-1.85 (m, 4H), 1.70 (A₂B₂, t, 2H), 1.63-1.40 (m, 3H).

### Example 3: Synthesis of tert-Butyl N⁶-(3-(4-iodophenyl)propanethioyl)-L-lysinate

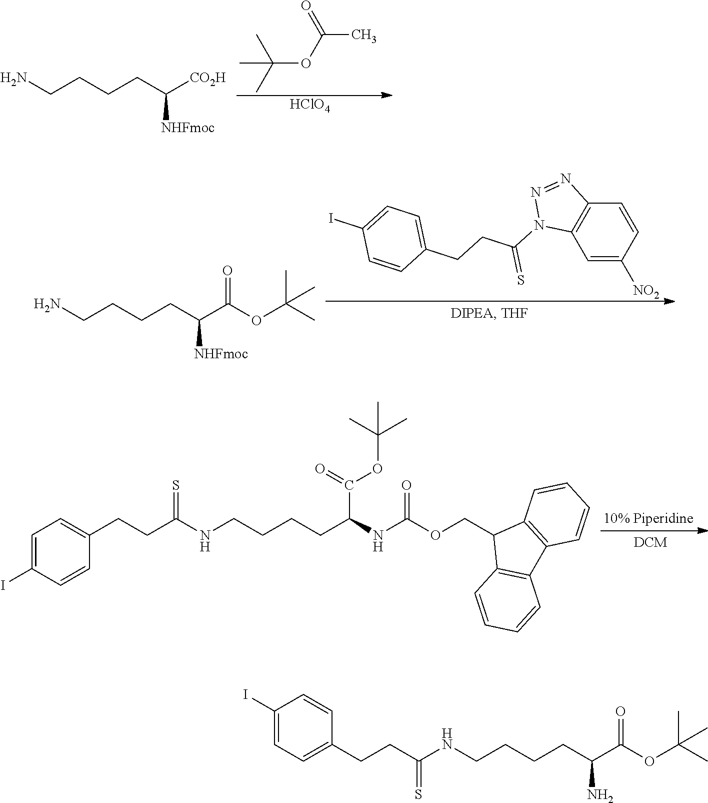

### Synthesis of tert-Butyl (((9H-fluoren-9-yl)methoxy)carbonyl)-L-lysinate

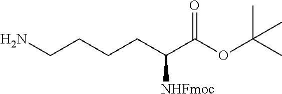

(((9H-Fluoren-9-yl)methoxy)carbonyl)-L-lysine (3 g, 8.14 mmol) was dissolved in tert-butyl acetate (42 ml) and cooled to −10° C. Perchloric acid (1.05 ml, 12.21 mmol, 1.5 eq) was added dropwise. After 4 h 200 mL EtOAc and 100 mL deionized water were added, and the mixture was partitioned in a separatory funnel. The organic layer was collected, dried of sodium sulfate, filtered, and evaporated under reduced pressure. The product was purified by silica gel column chromatography using 100% EtOAc as the eluant. Evaporation of the product fractions yielded a pale yellow free flowing powder. ¹H NMR (400 MHz, CD3Cl) δ 7.71 (d, J=7.5 Hz, 2H), 7.58 (d, J=7.9 Hz, 2H), 7.35 (t, 2H, J=7.4 Hz,), 7.28 (d, J=5.8 Hz, 2H), 5.52 (d, J=7.96 Hz, 1H), 4.32 (t, 1H), 4.37 (t, 1H), 3.12 (t, 2H), 1.41 (s, 9H).

### Synthesis of tert-Butyl N²-(((9H-fluoren-9-yl) methoxy)carbonyl)-N⁶-(3-(4-iodophenyl)propanethioyl)-L-lysinate

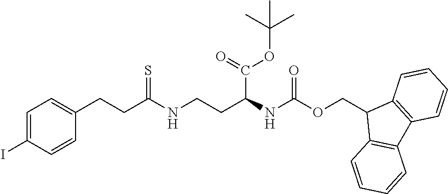 [SMILES: CC(C)(C)OC(=O)[C@H](CCNC(=S)CCc1ccc(I)cc1)NC(=O)OCC1c2ccccc2-c2ccccc21]

Under an atmosphere of dry nitrogen, a solution of 3-(4-iodophenyl)-1-(6-nitro-1H-benzo[d][1,2,3]triazol-1-yl)propane-1-thione (4.7 mmol, 2.05 g) in 50 mL of THF was treated with tert-butyl (((9H-fluoren-9-yl)methoxy)carbonyl)-L-lysinate (2.06 g, 4.7 mmol). The mixture was cooled to 0° C. and DIPEA (4.2 mmol, 0.7 mL) was added dropwise. After the addition was complete, the mixture was allowed to stir overnight at room temperature. The mixture was neutralized with 1M HCl and transferred to a separatory funnel containing EtOAc and deionized water. The organic fraction was isolated, dried over sodium sulfate, filtered, and concentrated under reduced pressure. The residue was purified by silica gel column chromatography (10% MeOH in CH₂Cl₂) to afford the titled compound in 70% yield. ¹H NMR (400 MHz, CDCl³) δ 7.78 (d, J=7.56 Hz, 2H), 7.57 (d, J=7.4 Hz, 2H), 7.50 (d, J=7.96 Hz, 2H), 7.44 (t, J=8.16 Hz, 2H), 7.33 (t, J=7.52 Hz, 2H), 6.89 (d, J=7.96 Hz, 2H), 5.48 (d, J=7.92 Hz, 1H), 4.41 (m, 1H), 4.33 (t, J=7.32 Hz, 1H), 4.23 (m, 2H), 3.56 (m, 2H), 3.04 (m, 2H), 2.81 (t, J=7.52 Hz, 2H), 1.82 (m, 1H), 1.66 (m, 2H), 1.50 (s, 9H), 1.38 (m, 2H).

### Synthesis of tert-Butyl N⁶-(3-(4-iodophenyl)propanethioyl)-L-lysinate

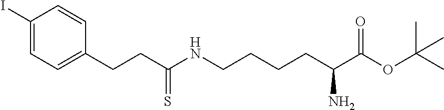 [SMILES: CC(C)(C)OC(=O)[C@@H](N)CCCCNC(=S)CCc1ccc(I)cc1]

To a solution of tert-butyl N²-(((9H-fluoren-9-yl)methoxy)carbonyl)-N⁶-(3-(4-iodophenyl)propanethioyl)-L-lysinate (1.43 mmol, 1.0 g) in 15 mL of CH₂Cl₂ under N₂ was added a 10% solution of piperidine in CH₂Cl₂ (˜40 eq). The mixture was allowed to stir overnight at room temperature (although an in-process TLC indicated the reaction was complete in the first 4 h). The solvent was evaporated under reduced pressure and the residue was purified by silica gel column chromatography (50% EtOAc in hexanes). Evaporation of the product fractions gave and off-white powder in 86% yield (0.51 g). ¹H NMR (400 MHz, CDCl₃) δ 7.66 (s (broad), 1H), 7.61 (d, J=8.24 Hz, 2H), 6.98 (d, J=8.24 Hz, 2H), 3.58 (m, 2H), 3.28 (dd, J=7.94, 5.08 Hz, 1H), 3.09 (t, J=7.32 Hz, 2H), 2.87 (t, J=7.32 Hz, 2H), 1.72 (m, 1H), 1.58 (t, J=7.20 Hz, 2H), 1.48 (s, 9H), 1.38 (m, 2H).

### Example 4: Additional Synthesis of Thioamide Containing Compositions

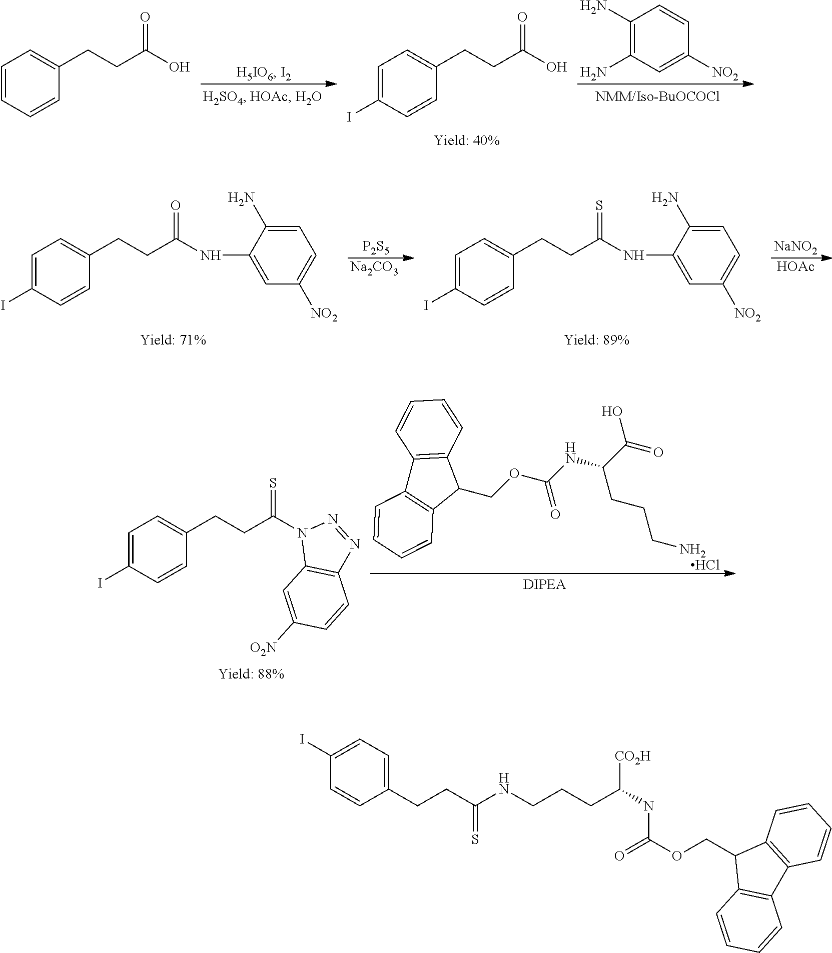

### Synthesis of N-(2-amino-5-nitrophenyl)-(4-iodophenyl)propanamide

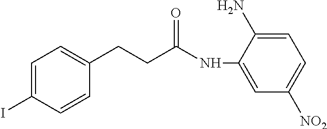 [SMILES: Nc1ccc([N+](=O)[O-])cc1NC(=O)CCc1ccc(I)cc1]

At 20° C. under N₂ carbonyldiimidazole (CDI, 8.8 mL, 80 mmol, 2.0 eq) was added to a THF solution (200 mL) of 3-(4-iodophenyl)propanoic acid (16.44 g, 40 mmol). Isobutyl chloroformate (5.2 mL, 40 mmol, 1.0 eq) was added was added dropwise and the reaction mixture was stirred for 30 min. A solution of 4-nitro-1,2-phenylenediamine (6.12 g, 40 mmol, 1.0 eq) in THF (100 mL) was added, and the mixture was stirred for a further 1.5 h at −20° C. and 15 h at 23° C. The mixture was filtered, and the filtrate was evaporated to dryness. The residue was dissolved in EtOAc (300 mL) and washed with aqueous solutions of 1 M NaH₂PO₄ (2×100 mL), saturated brine (2×100 mL), saturated NaHCO₃ (2×100 mL), and saturated NaCl (2×100 mL), dried over Na₂SO₄, and evaporated to dryness. The crude product was sonicated in EtOAc until it solidified. The EtOAc was decanted away and the remaining solid was filtered and evaporated in vacuo to yield the title compound (10.77 g, 63%) as a yellow-brown solid. ¹H NMR (400 MHz, DMSO-d₆) δ 9.19 (s, 1H), 8.20 (d, J=2.6 Hz, 1H), 7.84 (dd, J=7.8, 2.6 Hz, 1H), 7.64 (d, J=8.2 Hz, 2H), 7.10 (d, J=8.2 Hz, 2H), 6.75 (d, J=9.04 Hz, 1H), 6.43 (s (br), 2H), 2.88 (t, J=7.3 Hz, 2H), 2.66 (t, J=8.04 Hz, 2H).

### Synthesis of N-(2-amino-5-nitrophenyl)-3-(4-iodophenyl)propanethioamide

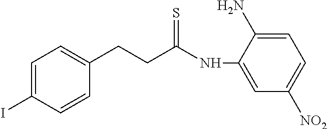 [SMILES: Nc1ccc([N+](=O)[O-])cc1NC(=S)CCc1ccc(I)cc1]

P₂S₅ (4.44 g, 20 mmol, 1.0 eq) was added to a suspension of Na₂CO₃ (1.08 g, 10 mmol, 0.5 eq) in THF (200 mL) at 23° C. under a flow of N₂. After 1 h, the mixture was cooled to 0° C. and N-(2-amino-5-nitrophenyl)-3-(4-iodophenyl)propanamide (8.22 g, 20 mmol) in THF (100 mL) was added dropwise and the mixture was allowed to stir for 2 h at 0° C. followed by 1 h at 23° C. The solvent was evaporated, and the residue was dissolved in EtOAc (200 mL), washed with 5% aqueous NaHCO₃ (2×100 mL). The aqueous phase was extracted with additional EtOAc (100 mL) and the combined organic phases were dried over Na₂SO₄, filtered and evaporated. The crude product was sonicated in EtOAc until it solidified. The EtOAc was decanted away and the remaining solid was filtered and dried in vacuo to yield the titled compound (6.89 g, 76%) as a yellow solid. ¹H NMR (400 MHz, DMSO-d6) δ 11.0 (s (broad), 1H), 7.84 (dd, J=9.08, J=2.4 Hz, 1H), 7.84 (d, J=2.64 Hz, 1H), 7.67 (d, J=8.24 Hz, 2H), 7.12 (d, J=8.28, 2H), 6.77 (d, J=9.12 Hz, 1H), 6.43 (s (broad), 2H), 3.04 (m, 2H), 3.06 (m, 2H).

### Synthesis of 3-(4-Iodophenyl)-1-(6-nitro-1H-benzo[d][1,2,3]triazol-1-yl)propane-1-thione

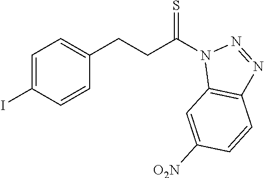 [SMILES: O=[N+]([O-])c1ccc2nnn(C(=S)CCc3ccc(I)cc3)c2c1]

N-(2-Amino-5-nitrophenyl)-3-(4-iodophenyl)propanethioamide (5.46 g, 12.8 mmol) was warmed to 40° C. in 95% glacial acetic acid diluted with 5% water (300 mL) and then cooled to 0° C. NaNO₂ (1.32 g, 19.2 mmol, 1.5 eq) was added in portions to the stirred solution over 20 min. After 30 min, the precipitated product was filtered, washed with water, and the filtrate was extracted with EtOAc (2×150 mL).

The combined organic phases were washed successively with H₂O (3×100 mL), saturated NaHCO₃ (2×100 mL), and brine (2×100 mL). The organic phase was separated, dried over Na₂SO₄, filtered and was evaporated. The solid so obtained was sonicated in a small amount of EtOAc (5 mL) and mixture was filtered to obtain the product as a yellow solid (3.23 g, 56%), which was dried under dynamic vacuum. ¹H NMR (400 MHz, CDCl3) δ 9.73 (d, J=1.96 Hz, 1H), 8.47 (dd, J=8.9, 2.08 Hz, 1H), 8.33 (d, J=8.9 Hz, 1H), 7.64 (d, J=8.32 Hz, 2H), 7.06 (d, J=8.28 Hz, 2H), 4.08 (t, J=7.64 Hz, 2H), 3.28 (t, J=7.92 Hz, 2H).

### Synthesis of N²-(((9H-fluoren-9-yl)methoxy)carbonyl)-N⁶-(3-(4-iodophenyl)propanethioyl)-D-lysine

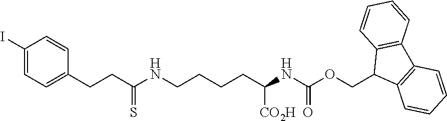 [SMILES: O=C(N[C@H](CCCCNC(=S)CCc1ccc(I)cc1)C(=O)O)OCC1c2ccccc2-c2ccccc21]

To a solution of 3-(4-iodophenyl)-1-(6-nitro-1H-benzo[d][1,2,3]triazol-1-yl)propane-1-thione (0.46 mmol, 200.0 mg) in 4 mL of THF was added Fmoc-L-Lys (0.46 mmol, 169.5 mg). The mixture was cooled to 0° C. before DIPEA (0.46 mmol, 81 μl) was added dropwise. The mixture was allowed to stir at room temperature for 12 h before it was neutralized with 1M HCl and diluted with deionized water. The mixture was extracted with EtOAc, dried over Na₂SO₄, filtered and was evaporated. The residue was purified by chromatography (10% MeOH in CH₂Cl₂) afforded the titled compound in 20% yield. ¹H NMR (400 MHz, DMSO-d6) δ 10.03 (s, 1H), 7.88 (d, J=7.48 Hz, 2H), 7.69 (d, J=7.4 Hz, 2H), 7.60 (d, J=8.16 Hz, 2H), 7.40 (t, J=7.36 Hz, 2H), 7.32 (t, J=7.44 Hz, 2H), 7.02 (d, J=8.12 Hz, 2H), 6.90 (s, 1H), 4.29 (m, 1H), 4.21 (m, 2H), 3.77 (m, 1H), 2.92 (t, J=8.12 Hz, 2H), 2.77 (t, J=8.16 Hz, 2H), 2.92 (t, J=8.12 Hz, 2H), 1.70 (m, 1H), 1.58 (m, 1H), 1.48 (m, 2H), 1.27 (m, 2H).

### Example 5. Suitable Lysine Derivatives

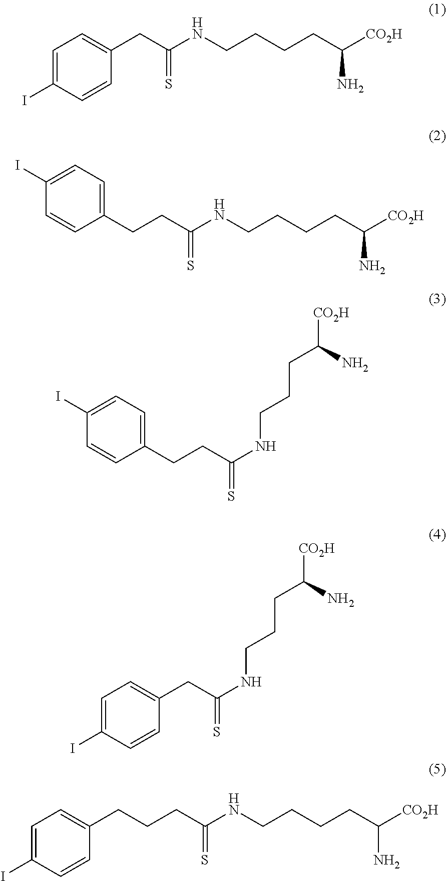 [SMILES: N[C@@H](CCCCNC(=S)Cc1ccc(I)cc1)C(=O)O ; N[C@@H](CCCCNC(=S)CCc1ccc(I)cc1)C(=O)O ; N[C@@H](CCCNC(=S)CCc1ccc(I)cc1)C(=O)O ; N[C@@H](CCCNC(=S)Cc1ccc(I)cc1)C(=O)O ; NC(CCCCNC(=S)CCCc1ccc(I)cc1)C(=O)O]

### Example 6. Stability of Thioamides to Hydrolysis

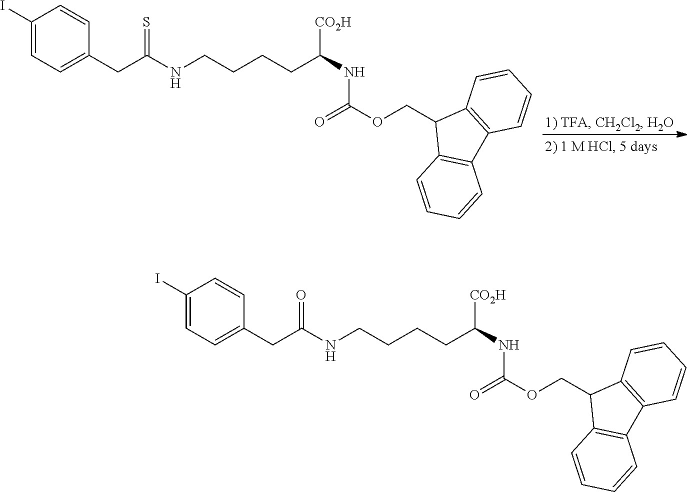 [SMILES: O=C(N[C@@H](CCCCNC(=S)Cc1ccc(I)cc1)C(=O)O)OCC1c2ccccc2-c2ccccc21 ; O=C(Cc1ccc(I)cc1)NCCCC[C@H](NC(=O)OCC1c2ccccc2-c2ccccc21)C(=O)O]

### Example 7. General Procedures of Forming Bioconjugates Featuring Thioamide Albumin Binding Groups

### Synthesis of tert-Butyl N²-(1-(9H-fluoren-9-yl)-3-thioxo-2,7,10-trioxa-4-azatridecan-13-oyl)-N⁶-(3-(4-iodophenyl)propanethioyl)-L-lysinate

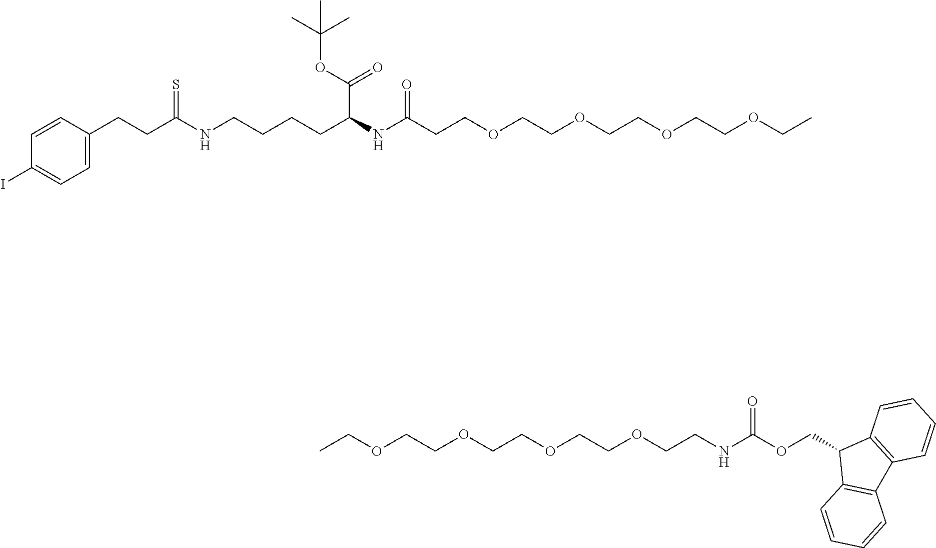

In a flame-dried flask under dry nitrogen, tert-butyl N⁶-(3-(4-iodophenyl)propanethioyl)-L-lysinate (0.200 g, 0.420 mmol, 1 eq) was dissolved in 2 mL of dry methylene chloride and set to stir. Anhydrous triethylamine (5.90 uL, 0.042 mmol, 0.1 eq) added by syringe followed by a solution of Fmoc-PEG8-NHS ester (0.383 g, 0.504 mmol, 1.2 eq) dissolved in 1 mL of dry methylene chloride. The mixture was allowed to stir 4 h hours at room temperature before the solvent was evaporated to leave a light-yellow syrup. The residue was purified by silica gel flash column chromatography (10% MeOH/DCM) to yield the title compound as a light-yellow syrup (0.188 g, 40%).

### Synthesis of tert-Butyl N²-(1-(9H-fluoren-9-yl)-3-oxo-2,7,10,13,16,19,22,25,28-nonaoxa-4-azahentriacontan-31-oyl)-N⁶-(3-(4-iodophenyl)propanethioyl)-L-lysinate

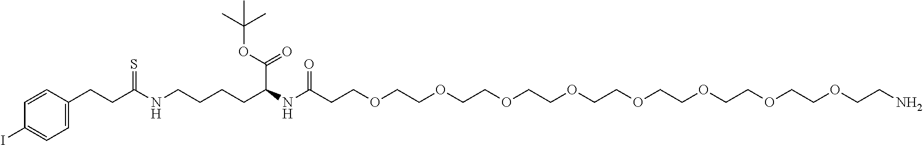 [SMILES: CC(C)(C)OC(=O)[C@H](CCCCNC(=S)CCc1ccc(I)cc1)NC(=O)CCOCCOCCOCCOCCOCCOCCOCCOCCN]

tert-Butyl N²-(1-(9H-fluoren-9-yl)-3-thioxo-2,7,10-trioxa-4-azatridecan-13-oyl)-N⁶-(3-(4-iodophenyl)propanethioyl)-L-lysinate (0.188 g, 0.168 mmol) was dissolved in a 10% piperidine solution in dry methylene chloride (5 mL). The mixture was allowed to stir for 4 h hours at room temperature before the solvent was removed to leave a light-yellow powder. The residue was purified by silica gel flash column chromatography (5% MeOH/DCM) to yield a light-yellow syrup (0.133 g, 88%).

### Synthesis of tert-Butyl N2-((S)-10-((((9H-fluoren-9-yl)methoxy)carbonyl)amino)-2,2-dimethyl-4,11-dioxo-3,15,18,21,24,27,30,33,36-nonaoxa-5,12-diazanonatriacontan-39-oyl)-N6-(3-(4-iodophenyl)propanethioyl)-L-lysinate

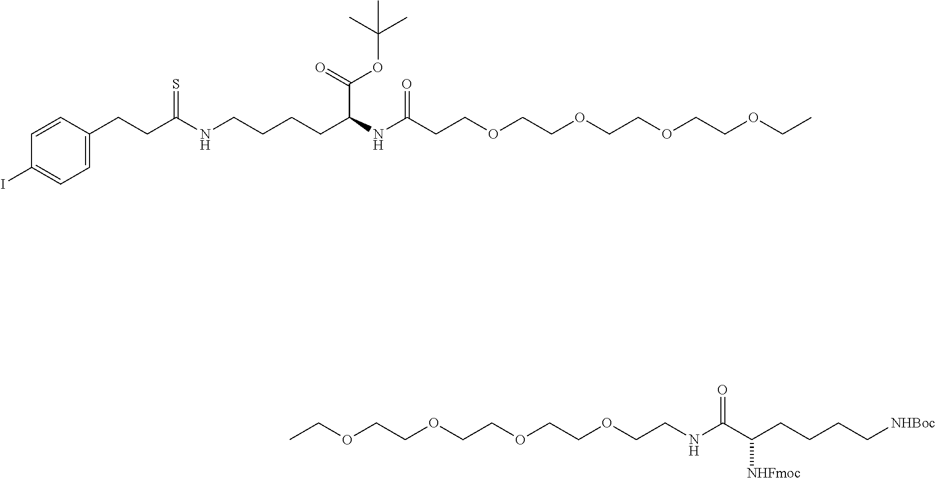

In a flame-dried flask under nitrogen tert-butyl N²-(1-(9H-fluoren-9-yl)-3-oxo-2,7,10,13,16,19,22,25,28-nonaoxa-4-azahentriacontan-31-oyl)-N⁶-(3-(4-iodophenyl)propanethioyl)-L-lysinate (0.133 g, 0.147 mmol, 1 eq) was dissolved in 3 mL dry methylene chloride. Dry triethylamine (20.50 uL, 0.147 mmol, 1 eq) added to the stirred solution, followed by a solution of Boc-Lys(Boc)-OSu (71.71 mg, 0.161 mmol, 1.1 eq) in 3 mL dry methylene chloride. The mixture was allowed to stir for 4 h hours at room temperature before the solvent was removed under reduced pressure to leave a yellow solid. The residue was purified by silica gel flash column chromatography (5% MeOH/DCM) to yield a yellow syrup (0.139 g, 70%).

### Synthesis of tert-Butyl N2-((S)-10-amino-2,2-dimethyl-4,11-dioxo-3,15,18,21,24,27,30,33,36-nonaoxa-5,12-diazanonatriacontan-39-oyl)-N6-(3-(4-iodophenyl)propanethioyl)-L-lysinate

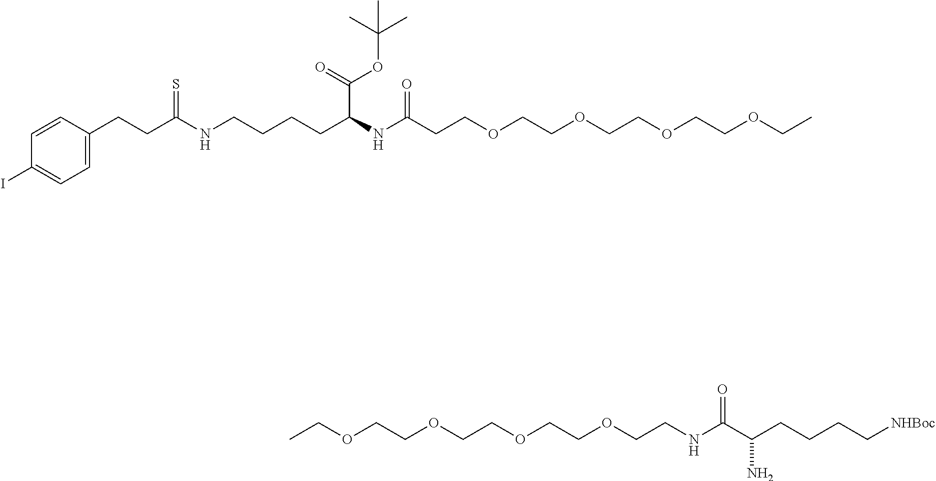

tert-Butyl N2-((S)-10-((((9H-fluoren-9-yl)methoxy)carbonyl)amino)-2,2-dimethyl-4,11-dioxo-3,15,18,21,24,27,30,33,36-nonaoxa-5,12-diazanonatriacontan-39-oyl)-N6-(3-(4-iodophenyl)propanethioyl)-L-lysinate (0.139 g, 0.103 mmol) was treated with a solution of piperidine (10%) in dry methylene chloride 5 mL. The mixture was allowed to stir for 4 h hours at room temperature before the solvent was removed under reduced pressure to leave a light-yellow powder. The residue was purified by silica gel flash column chromatography (5% MeOH/DCM) to yield a light-yellow syrup (0.093 g, 80%).

### Synthesis of N6-(3-(4-iodophenyl)propanethioyl)-N2-(17-oxo-21-((3aR,4R,6aS)-2-oxohexahydro-1H-thieno[3,4-d]imidazol-4-yl)-4,7,10,13-tetraoxa-16-azahenicosanoyl)-L-lysine

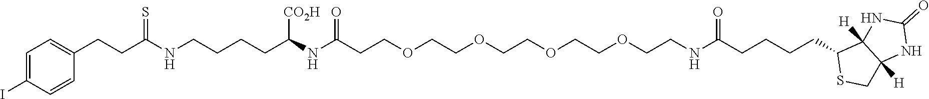 [SMILES: O=C(CCCC[C@H]1SC[C@H]2NC(=O)N[C@H]21)NCCOCCOCCOCCOCCC(=O)N[C@@H](CCCCNC(=S)CCc1ccc(I)cc1)C(=O)O]

(S)-1-carboxy-5-(3-(4-iodophenyl)propanethioamido)pentan-1-aminium trifluoroacetate salt (0.100 g, 0.187 mmol, 1 eq) was dissolved in 1 mL DMF (dimethylformamide) and triethylamine (52.20 uL, 0.374 mmol, 2 eq) was added. When triethylammonium trifluoroacetate began to precipitate, a potassium carbonate buffer (pH=8) solution was added dropwise to form a homogeneous solution. Subsequently, NHS-PEG4-Biotin (0.121 g, 0.205 mmol, 1.1 eq) dissolved in 1 mL DMF and added to the solution and the mixture was allowed to stir for 8 h. Deionized water (2 mL) and methylene chloride (5 mL) were added and the organic layer was washed with water (3×5 mL) to remove DMF. The organic layer was evaporated, and the colorless, powdery residue was purified by silica gel flash column chromatography (5% MeOH/DCM) to yield an off-white powder (0.100 g, 60%).

Those skilled in the art will recognize, or be able to ascertain using no more than routine experimentation, many equivalents to the specific embodiments of the invention described herein. Where any concept(s) or element(s) of the invention is separately presented for convenience, it is understood that the combination of any such separately presented concept(s) or element(s), as necessary, is also encompassed by the invention. Such equivalents are intended to be encompassed by the claims.

The contents of the patents and references cited throughout this specification are hereby incorporated by reference in their entireties.

We claim:

1) A compound of Formula (I): 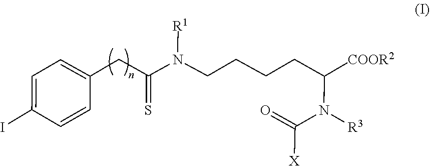
or a pharmaceutically acceptable salt thereof;
wherein:
R¹ is H, C₁-C₆ alkyl, or a protecting group;
R² is H, C₁-C₆ alkyl, or a protecting group;
R³ is H, C₁-C₆ alkyl, or a protecting group;
X is a therapeutic drug; and
n is 0, 1, 2, 3, 4, or 5.

2) The compound of claim 1, wherein n is 2 or 3.

3) The compound of claim 1, wherein the compound of Formula (I) is a compound of Formula (II): 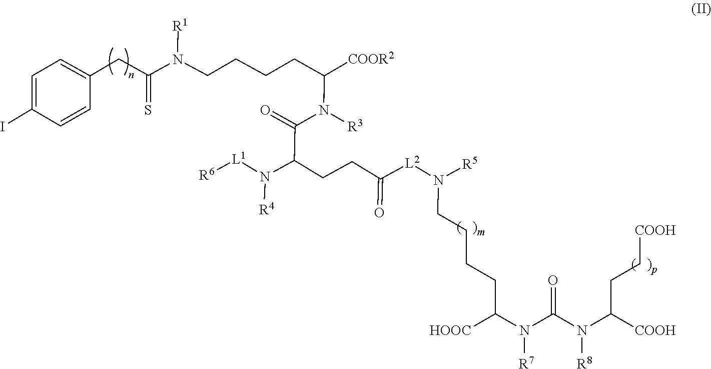
or a pharmaceutically acceptable salt thereof;
wherein:
R¹ is H, C₁-C₆ alkyl, or a protecting group;
R² is H, C₁-C₆ alkyl, or a protecting group;
R³ is H, C₁-C₆ alkyl, or a protecting group;
L¹ is a natural amino acid, an unnatural amino acid, or (X)_{q}—(Y)ᵣ—(Z)ₛ, wherein X is C₁-C₃₀ alkyl, Y is C₁₀-C₃₀ heteroaromatic, and Z is C₁-C₁₂ alkyl, wherein any of the methylene groups in the alkyl group of L¹ may be replaced with —O—, NH, or carbonyl;
R⁴ is H, C₁-C₆ alkyl, or a protecting group;
R⁵ is H, C₁-C₆ alkyl, or a protecting group;
R⁶ is a therapeutic drug or chelating agent;
R⁷ is H, C₁-C₆ alkyl, or a protecting group;
R⁸ is H, C₁-C₆ alkyl, or a protecting group;
L² is a bond, —N(R⁹)—C₁-C₁₂ alkyl-C(O)—, —N(R⁹)—C₄-C₃₀ alkylcycloalkyl-C(O)—C₇-C₃₀ alkylaryl-C(O)—, or —N(R⁹)—C₇-C₃₀ alkylaryl-C(O)NH—C₇-C₃₀ alkylaryl-C(O)NH—CH(CO₂H)—C₁-C₁₂ alkyl-NHC(O)—C₁-C₁₂ alkyl-C(O)—, wherein C₇-C₃₀ alkylaryl is optionally substituted with halo or hydroxyl;
n is 0, 1, 2, 3, 4, or 5;
m is 0, 1, 2, 3, 4, or 5;
p is 0, 1, 2, 3, 4, or 5;
q is 0 or 1;
r is 0 or 1; and
s is 0 or 1.

4) The compound of claim 3, wherein n is 3.

5) The compound of claim 3, wherein L¹ is X—Y—Z, and wherein:
X is 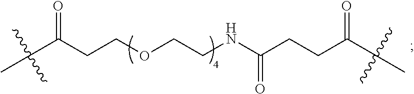
Y is 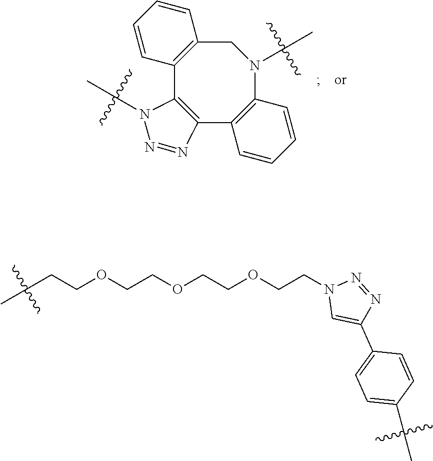 [SMILES: *N1Cc2ccccc2-c2c(nnn2*)-c2ccccc21 ; *CCOCCOCCOCCn1cc(-c2ccc(*)cc2)nn1]
Z is C₁-C₁₂ alkyl, wherein any of the methylene groups in the alkyl group may be replaced with NH or carbonyl.

6) The compound of claim 3, wherein L¹ is Z, and wherein:
Z is C₁-C₁₂ alkyl, wherein any of the methylene groups in the alkyl group may be replaced with NH or carbonyl.

7) The compound of claim 6, wherein Z is

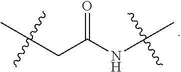 [SMILES: *CC(=O)N*]

8) The compound of claim 1, wherein the chelating agent is

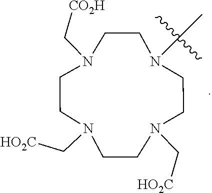 [SMILES: *N1CCN(CC(=O)O)CCN(CC(=O)O)CCN(CC(=O)O)CC1]

9) The compound of claim 1, wherein L² is —N(R⁹)—C₁-C₁₂ alkyl-C(O)—.

10) The compound of claim 9, wherein L² is

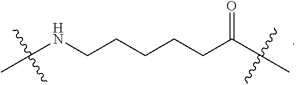 [SMILES: *NCCCCCC(*)=O]

11) The compound of claim 1, wherein L² is —N(R⁹)—C₄-C₃₀ alkylcycloalkyl-C(O)—C₇-C₃₀ alkylaryl-C(O)—.

12) The compound of claim 12, wherein L² is

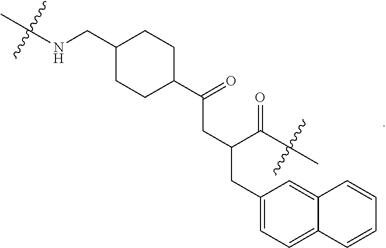 [SMILES: *NCC1CCC(C(=O)CC(Cc2ccc3ccccc3c2)C(*)=O)CC1]

13) The compound of claim 1, wherein L² is —N(R⁹)—C₇-C₃₀ alkylaryl-C(O)NH—C₇-C₃₀ alkylaryl-C(O)NH—CH(CO₂H)—C₁-C₁₂alkyl-NHC(O)—C₁-C₁₂alkyl-C(O)—, wherein C₇-C₃₀ alkylaryl is optionally substituted with halo or hydroxyl.

14) The compound of claim 13, wherein L² is

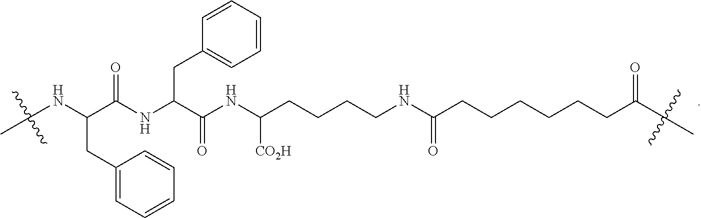 [SMILES: *NC(Cc1ccccc1)C(=O)NC(Cc1ccccc1)C(=O)NC(CCCCNC(=O)CCCCCCC(*)=O)C(=O)O]

15) The compound of claim 13, wherein L² is

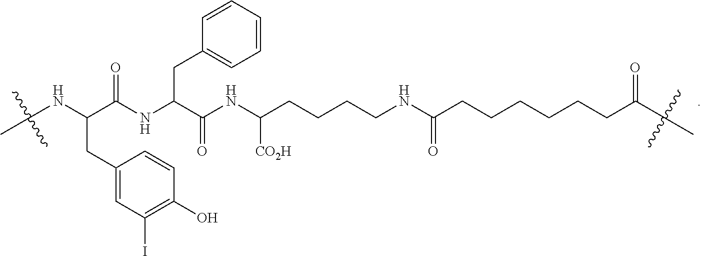 [SMILES: *NC(Cc1ccc(O)c(I)c1)C(=O)NC(Cc1ccccc1)C(=O)NC(CCCCNC(=O)CCCCCCC(*)=O)C(=O)O]

16) The compound of claim 1, wherein the compound of Formula (I) is a compound of Formula (III): 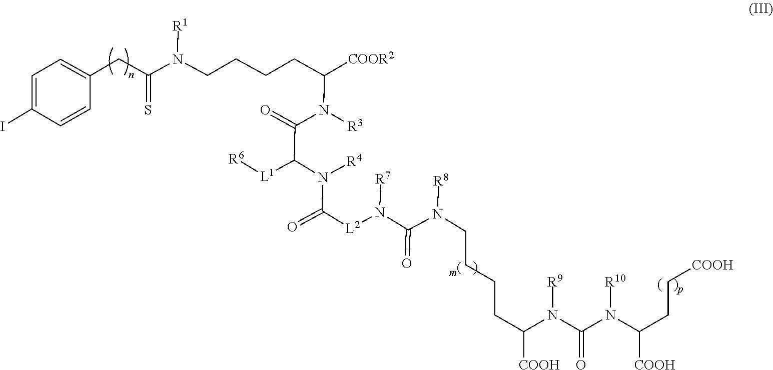
or a pharmaceutically acceptable salt thereof;
wherein:
R¹ is H, C₁-C₆ alkyl, or a protecting group;
R² is H, C₁-C₆ alkyl, or a protecting group;
R³ is H, C₁-C₆ alkyl, or a protecting group;
L¹ is a natural amino acid, an unnatural amino acid, or (X)_{q}—(Y)ᵣ—(Z)ₛ, wherein X is C₁-C₂₀ alkyl, Y is C₁₀-C₃₀ aryl, and Z is C₁-C₁₂ alkyl, wherein any of the methylene groups in the alkyl group of L¹ may be replaced with —O—, NH, carbonyl, or thiocarbonyl;
R⁴ is H, C₁-C₆ alkyl, or a protecting group;
R⁵ is H, C₁-C₆ alkyl, or a protecting group;
R⁶ is a therapeutic drug or chelating agent;
R⁷ is H, C₁-C₆ alkyl, or a protecting group;
R⁸ is H, C₁-C₆ alkyl, or a protecting group;
R⁹ is H, C₁-C₆ alkyl, or a protecting group;
R¹⁰ is H, C₁-C₆ alkyl, or a protecting group;
L² is C₁-C₃₀ alkyl-C₃-C₁₈ heteroaryl-C₆-C₁₈ aryl, wherein any of the methylene groups in the alkyl group may be replaced with —O—;
n is 0, 1, 2, 3, 4, or 5;
m is 0, 1, 2, 3, 4, or 5;
p is 0, 1, 2, 3, 4, or 5;
q is 0 or 1;
r is 0 or 1; and
s is 0 or 1.

17) The compound of claim 16, wherein n is 3.

18) The compound of claim 16, wherein L¹ is X—Y—Z, and wherein:
X is 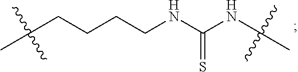 [SMILES: *CCCCNC(=S)N*]
Y is C₁₀-C₃₀ aryl; and
Z is C₁-C₁₂ alkyl, wherein any of the methylene groups in the alkyl group may be replaced with NH or carbonyl.

19) The compound of claim 16, wherein the chelating agent is

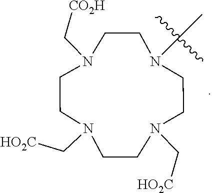 [SMILES: *N1CCN(CC(=O)O)CCN(CC(=O)O)CCN(CC(=O)O)CC1]

20) The compound of claim 16, wherein L² is

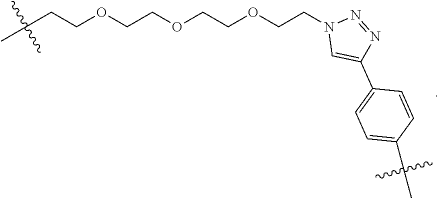 [SMILES: *CCOCCOCCOCCn1cc(-c2ccc(*)cc2)nn1]

21) A compound of Formula (IV): 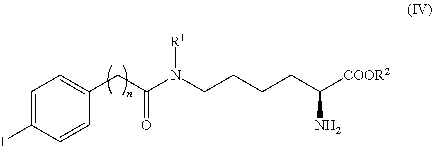
or a pharmaceutically acceptable salt thereof;
wherein:
R¹ is H, C₁-C₆ alkyl, or a protecting group;
R² is H, C₁-C₆ alkyl, or a protecting group; and
n is 0, 1, 2, 3, 4, or 5.

22) The compound of claim 1, wherein n is 2 or 3.
